(19) United States
(12) Patent Application Publication
(10) Pub. No.: US 2007/0128693 A1
(43) Pub. Date: Jun. 7, 2007

(54) Method for the inactivation and removal of dengue virus from biological samples

(75) Inventors: Bing Lou Wong, Hong Kong (HK); Yiwu Xie, Hong Kong (HK)

Correspondence Address: EAGLE IP LIMITED, UNIT 1201, 12/F KWAI HUNG HOLDINGS CENTRE, 89 KING'S ROAD, NORTH POINT, HONG KONG, CN

(73) Assignee: Advantek Serum Laboratories Limited3/F, Shatin, N.T. Hong Kong (CN)

(21) Appl. No.: 11/294,432

(22) Filed: Dec. 6, 2005

(51) Int. Cl. C12P 21/06; C12N 7/00; C12N 7/01

(52) U.S. Cl. 435/69.1; 435/235.1

(57) ABSTRACT

A method for inactivating and removing dengue virus from a biological sample comprising the dengue virus and at least one biomolecule of interest, wherein a solvent treatment step is used to inactivate the dengue virus, then a cation exchange chromatography step effectively removes the dengue virus by physically separating it from the biomolecule of interest. In another aspect of the present invention, a method is provided for producing human plasma albumin that is substantially free from infective dengue virus.

### FIELD OF INVENTION

This invention relates to the inactivation and removal of viruses, in particular the inactivation and removal of the dengue virus from a biological sample.

### BACKGROUND OF INVENTION

Globally, dengue virus infects 50 to 100 million people a year, and the mosquito-borne sickness is steadily on the rise. Dengue virus is known to survive over long periods in fluids with high protein contents, e.g. blood. Therefore, dengue virus may be transmitted via transfusion of blood or blood components. This becomes a serious public health problem without proper control measures.

U.S. Pat. No. 5,808,011 discloses a method for chromatographic removal of prions. U.S. Pat. No. 6,468,733 discloses a method of the inactivation of viruses by a solvent/detergent (S/D) combination and nanofiltration. U.S. patent application Ser. No. 10/220,929 discloses a method of producing IgG comprising adsorbing IgG to a cation exchanger and collecting the adsorbed IgG fraction, which may be virus contaminated and requires subsequent virus inactivation.

The transmission of blood-borne pathogens such as the dengue virus is of particular concern in the manufacture of whole blood or plasma derived medicinal products. A starting batch of blood containing a single contaminated unit of plasma can potentially transmit a blood-borne disease to a large number of recipients.

It is thus an object of the present invention to provide a method of dengue virus inactivation and removal from biological samples.

### SUMMARY OF INVENTION

Accordingly, the present invention, in one aspect, is a method for inactivating and removing dengue virus from a biological sample containing at least one biomolecule of interest, such method comprising the steps of: (i) treating the biological sample with at least one solvent under conditions sufficient to inactivate dengue viruses and (ii) separating said virus from said biomolecule by cation exchange chromatography.

The solvent in step (i) is used to deactivate lipid coated viruses and such solvent may be used in combination with a nonionic detergent. Step (i) above is preferably performed using tri-(n-butyl)phosphate at a concentration of 0.1%-2.0% as the solvent, in combination with octylphenoxypolyethoxyethanol nonionic surfactant at a concentration of 0.1%-5.0%. The solvent in step (i) may also be tri-(t-butyl)phosphate, tri-(n-hexyl)phosphate, tri-(2-ethylhexyl)phosphate, or tri-(n-decyl)phosphate. In this preferred embodiment, the treatment conditions of step (i) are 1° C.-50° C. for at least 1 hour.

Step (ii) above may be performed using any chromatography medium. Some non-limiting examples of such are of silica, alumina, titania, cross-linked dextran, agarose, cross-linked agarose or a polymer derivatized with a cationic group. In the preferred embodiment, the cation exchange chromatography uses a column medium of cross-linked agarose attached to carboxy methyl groups, and step (ii) comprises loading and equilibrating the column at pH 4.0±0.5, washing the column at pH 7.0±0.5, and eluting the biomolecule of interest using 0.1M glycine plus 0.15M NaCl at pH 9.0±0.5.

In the preferred embodiment, the biological sample is obtained from human plasma, a precipitate of human plasma, or a cryoprecipitate of human plasma, and the biomolecule of interest is immunoglobulin. The sequential steps used to obtain human immunoglobulin from human plasma are as follows: (i) precipitating the human plasma with cold ethanol precipitation using 8±0.5% ethanol at pH 7.1±0.1, followed by 19±0.5% ethanol at pH 5.85±0.05, (ii) re-dissolving the precipitate and adding NaAc/HAc buffer of about 0.8M/4M and pH 3.9, (iii) adjusting the pH to 5.1±0.1, (iv) adding ethanol to a final ethanol concentration of 15±1.0%, (v) mixing gently for 1.5±0.5 hours, at a temperature of −5° C. to −5.5° C., and (vi) centrifuging at 2,300×g to obtain the supernatant containing immunoglobulins.

In a variation of the above method, a further step of filtering with a 0.22 μm or 0.1 μm filter followed by ultrafiltrating with a 35 nm filter, comes before treating the biological sample with a solvent to inactivate dengue viruses, as in step (i) of paragraph [0006].

In the most preferred embodiment, the method for inactivating and removing dengue virus from an immunoglobulin solution is as follows: (i) the immunoglobulin solution is treated with a mixture of octylphenoxypolyethoxyethanol nonionic surfactant and tri-(n-butyl)phosphate, (ii) the virus is removed from the biomolecule of interest through cation exchange chromatography using a column comprising cross-linked agarose attached to carboxylmethyl groups and involving the removal steps of equilibrating the column to pH 4.0±0.1, washing the column with glycine at pH 7.0±0.1, and eluting the biomolecule of interest using 0.1M glycine plus 0.15M NaCl at pH 9.0±0.1.

In this most preferred embodiment, octylphenoxypolyethoxyethanol nonionic surfactant is used at a concentration of 1±0.1% and said tri-(n-butyl)phosphate is used at a concentration of 0.3±0.1%, and the immunoglobulin solution is treated for a duration of at least 1 hour, at 28-32° C. Such duration may be at least 4 hours, or preferably any duration in the range of 4 to 16 hours.

A further aspect of the present invention is a method for producing human plasma albumin that is substantially free from infective dengue virus. This method comprising the sequential steps of: (i) precipitating the human plasma with cold ethanol of the following concentrations and pHs, sequentially: 8±0.5% ethanol at pH 7.1±0.1, followed by 19±0.5% ethanol at pH 5.85±0.05, 40±0.5% ethanol at pH 5.86±0.05, and 40±0.5% ethanol at pH 4.77±0.05, (ii) adding 0.032M sodium caprylate of pH 6.8 to said albumin, and (iii) heating the resulting mixture to 59-61° C. for a duration of at least 1 hour

There are many advantages to the present invention. The foremost advantage being that the inventive ion exchange chromatography step removes both active and inactivated dengue virus and dengue virus RNA from the biological sample. Therefore, for example, any risk of undesired immunological response from introducing foreign virus or virus genetic materials, albeit inactivated, into the human recipient is eliminated. Moreover, each step in the present invention is optimized to inactivate and/or remove dengue virus and the said process as a whole provides a strong measure of confidence for removing the dengue virus, in both its active and inactivated forms, such that biological samples which have undergone the process of this invention meet safety standards, regarding the dengue virus, for human therapeutic use.

Thus, the cation exchange chromatography step in the present invention is a simple and cost-effective process for separating both virus and the solvent, or the solvent/detergent combination, from the biological sample in a single step.

### DETAILED DESCRIPTION OF THE PREFERRED EMBODIMENTS

As used herein and in the claims, “comprising” means including the following elements but not excluding others.

The present invention introduces a method for the inactivation and removal of dengue virus from biological samples. As used herein and in the claims, “biological sample” refers to any preparation obtained from a biological origin, for example, but not limited to, blood products, urine-derived products and cell lysate. “Blood products” refer to products of human or animal blood or plasma, such products being intended for therapeutic, prophylactic or diagnostic application. “Blood products” may contain, but are not limited to, enzymes, proenzymes including coagulation factors, enzyme inhibitors, immunoglobulin, albumin, plasminogen, fibrinogen, fibronectin or plasma.

As used in the present invention, the term “removal” refers to the removal of virus particles, proteins, or genetic materials, or any combinations thereof.

As used herein and in the claims, “Log reduction” refers to reduction by a factor of Log₁₀, unless otherwise stated.

In the preferred embodiment, the present invention comprises a method for the inactivation and/or removal of dengue virus via additional steps in the manufacture process of plasma proteins, said steps comprising virus filtration, solvent treatment and ion exchange chromatography for IgG production, and pasteurization for albumin production.

Specific examples 1 to 3 illustrate how the inventors practiced the above invention. Following examples 1 and 3 are descriptions of the quantification of dengue virus after using the inactivation and/or removal method of this invention. The quantification data show that dengue virus was successfully inactivated and/or removed.

### EXAMPLE 1

### The Inactivation and Removal of Dengue Virus in the Immunoglobulin Production Process

Step 1: Virus Filtration

Partially purified immunoglobulin solution underwent membrane filtration with either a 0.22 μm or a 0.1 μm filter (Millipore Steritop™, Massachusetts, US) to remove virus aggregates, respectively. The filtered immunoglobulin solution was subject to virus filtration with a Planova® 35N filter (Asahi Kasei, Tokyo, Japan) in a normal-flow manner under constant pressure of 80 kPa at ambient temperature.

Step 2: Solvent/Detergent Treatment

The immunoglobulin solution processed through virus filtration was heated to 28° C. Octylphenoxypolyethoxyethanol nonionic surfactant (i.e. Triton X-100) and tri-(n-butyl)phosphate (TnBP) were added drop-by-drop into the immunoglobulin solution to a final concentration of 1% and 0.3%, respectively. The time course of the solvent/detergent treatment is 16-hours at 28-32° C.

Step 3: Ion Exchange Chromatography

A chromatography column of 10-millimeter diameter was packed to a bed height of 11 centimeter with used CM Sepharose® Fast Flow resin (Pharmacia Biotech, Sweden) that had previously been re-cycled 476 times with the immunoglobulin purification process. The column was equilibrated with 0.02 M sodium acetate (NaAc) buffer, pH 4.0. Adjusted a pH of 4.0 with 1M HCl and an ionic strength of 1.4 mS/cm with purified water, the solvent/detergent treated immunoglobulin solution was applied to the column at a linear flow rate of 40 cm/h at ambient temperature. Following washing of the column with 10 column volumes of 0.01M glycine, pH 7.0, immunoglobulins were eluted with 0.1M glycine together with 0.15M NaCl, pH 9.0.

The preferred embodiments of the first aspect of the present invention are thus fully described. Although the description referred to particular embodiments, it will be clear to one skilled in the art that the present invention may be practiced with variation of these specific details. Hence this invention should not be construed as limited to the embodiments set forth herein.

Quantification of Dengue Virus in Example 1

Using the process described above, the following data shows that dengue virus was successfully inactivated and/or removed using the methods of the present invention.

In Example 1, human plasma fractions were spiked either with dengue virus at a ratio of 1:9-1:49 (vol/vol) or with porcine parvovirus at a ratio of 1:20-1:100 (vol/vol). After processing, test samples were taken for titration of virus. The results are shown in Table 1.

TABLE 1
Clearance of Viruses in the Immunoglobulin Process

Log Reduction of Virus

| Process step | Dengue Virus | Porcine Parvovirus |
|---|---|---|
| Fraction III Precipitation | 2.16 | — |
| Virus Filtration | ≧5.79 | 0.35 |
| S/D Treatment | ≧5.05 | — |
| Chromatography | ≧6.93 | 1.04 |
| Cumulative | ≧19.93 | 1.04^{#} |

Note:
^{#}virus reduction of less than 1 Log is not included in the ‘Cumulative’.

Before the filtration step, the biological sample was again spiked with dengue virus at a ratio of 1:10 (vol/vol). After the filtration step, samples were titrated for quantity of dengue virus or porcine parvovirus. The results are shown in Table 1.

In the subsequent step of solvent/detergent treatment, dengue virus was spiked at a ratio of 1:9 (vol/vol). Samples were taken out for virus titration during the time course of 16-hour treatment at 28-32° C. The solvent/detergent treatment step quickly inactivated dengue virus, and no dengue virus was detected after one minute of solvent/detergent treatment. Table 2 shows the Log reduction of dengue virus during the time course of this step.

TABLE 2
Inactivation of dengue virus during solvent/detergent treatment

| S/D Treatment | Log Reduction of Dengue Virus |
|---|---|
| 1-minute | ≧2.84 |
| 5-minute | ≧2.84 |
| 20-minute | ≧2.84 |
| 1-hour | ≧5.05 |
| 16-hour | ≧5.05 |

Note:
no virus was detected in the S/D-treated samples, difference in Log reduction numbers at different time points is a reflection of sample volume assayed.

Dengue viruses were successfully inactivated as seen from the results data in Table 1 and 2. However, although inactivated, the dengue virus remains in the biological sample. Therefore, the immunoglobulin solution is still contaminated with inactive dengue virus particles and dengue virus RNA. The cation exchange chromatography step, as described below, will also remove the dengue virus from the biological sample.

In the cation exchange chromatography, the biological sample was again spiked at a ratio of 1:20 with bovine viral diarrhea virus (BVDV), dengue virus (DV), hepatitis A virus (HAV), human immunodeficiency virus (HIV) or porcine parvovirus (PPV). To test the effectiveness of the cation exchange chromatography, the column load, the flow-through fraction, and the eluate fraction containing immunoglobulin, were titrated for quantity of viruses by 50% tissue culture infectious dose (TCID₅₀) or reverse transcriptase quantitative polymerase chain reaction (qRT-PCR) assay (see Table 3 and 4).

In the TCID₅₀ assay for BVDV, MDBK cells (American Type Culture Collection, Virginia, US) were seeded in 96-well plates in a volume of 150 μl per well. Each dilution of sample was added at 50 μl per well, and incubation was carried out at 36±2° C. with 5% CO₂. Plates were assessed for TCID₅₀ endpoint as cytopathic effect developed in the positive control. The TCID₅₀ endpoint was calculated according to the Spearman Kärber method.

For DV, Vero E6 cells (American Type Culture Collection, Virginia, US) were seeded in 96-well plates in a volume of 100 μl per well. Following one day incubation, 50 μl medium was added to each well. Each dilution of sample was added at 50 μl per well, and further incubation was carried out at 36±2° C. with 5% CO₂. Plates were assessed for TCID₅₀ endpoint as cytopathic effect developed on the 5^{th} day. The TCID₅₀ endpoint was calculated according to the Spearman Kärber method.

For HAV, FRHL-4 cells (Food and Drug Administration, Maryland, US) were seeded in 96-well plates, and each dilution of sample was added at 25 μl per well. Following an incubation of 70±10 minutes at 36±2° C. with 5% CO₂, plates were fed with 175 μl medium. Plates were assessed for TCID₅₀ endpoint as cytopathic effect developed in the positive control. The TCID₅₀ endpoint was calculated according to the Spearman Kärber method.

For HIV, C8166 cells (European Collection of Animal Cell Cultures, Wiltshire, UK) were added in 96-well plates in a volume of 50 μl per well. Each dilution of sample was added at 50 μl per well. During 10-day incubation at 36±2° C. with 5% CO₂, 50 μl medium was added to each well on two occasions, as required. Plates were assessed for TCID₅₀ endpoint as cytopathic effect developed on the 10^{th} day. The TCID₅₀ endpoint was calculated according to the Spearman Kärber method.

For PPV, PK-13 cells (American Type Culture Collection, Virginia, US) were seeded in 96-well plates, and each dilution of sample was added at 25 μl per well. Following an incubation of 70±10 minutes at 36±2° C. with 5% CO₂, plates were fed with 175 μl medium. Plates were assessed for TCID₅₀ endpoint as cytopathic effect developed in the positive control. The TCID₅₀ endpoint was calculated according to the Spearman Kärber method.

As for qRT-PCR, RNA of bovine viral diarrhea virus, dengue virus and human immunodeficiency virus type I was extracted in duplicate from samples 245 using QIAamp® Viral RNA Mini Kit (Qiagen, Cologne, Germany) according to the procedure provided by the manufacturer. Quantitative RT-PCR utilizing fluorogenic probe hydrolysis (TaqMan® developed by Applied Biosystems, California, US) technology was performed on samples and proper controls using primers and fluorescent probes specific for the viruses with conditions optimized 250 to detect 50 copies of viral RNA for BVDV and HIV, and 4.67 copies of viral RNA for DV. Triplicate PCR reactions were performed for each sample.

TABLE 3
Log Reduction of Viruses via Cation Exchange Chromatography

| | TCID₅₀ Assay (Log) | | | Log Reduction |
|---|---|---|---|---|
| Viruses | Load | Flow through | Eluate | Load → Eluate |
| BVDV | 6.36 | 4.35 | ≦0.64^{#} | ≧5.72 |
| DV | 6.17 | ≦4.32^{#} | ≦−0.76^{#} | ≧6.93 |
| HAV | 6.32 | 4.73 | ≦2.03^{#} | ≧4.29 |
| HIV | 6.11 | ≦3.98^{#} | ≦2.64^{#} | ≧3.47 |
| PPV | 7.46 | 7.22 | 6.42 | 1.04 |

Note:
^{#}indicates no virus detected, titer was calculated according to the Poisson distribution.

Our detailed experimental qRT-PCR results are shown in Table 4. The qRT-PCR results in Table 4 clearly show that removal of dengue virus RNA in the inventive cation exchange chromatography step is markedly more effective than that for the other viruses BVDV and HIV.

TABLE 4
Log Reduction of Viruses via Cation Exchange Chromatography

| | qRT-PCR Assay (Log copies) | | | Log Reduction |
|---|---|---|---|---|
| Viruses | Load | Flow through | Eluate | Load → Eluate |
| BVDV | 10.71 | 10.11 | 9.55 | 1.16 |
| DV | 7.55 | 6.66 | <2.52^{#} | >5.03 |
| HIV | 10.07 | 10.05 | 8.28 | 1.79 |

Note:
^{#}indicates no virus detected, the number was calculated using the detection limit of 4.67 copies/ml.

As listed in the Table 1, individual steps of the immunoglobulin process are very effective in the inactivation and removal of dengue virus, with a cumulative reduction of not less than 19.93 Log. In comparison, the immunoglobulin process is only mildly effective in the removal of porcine parvovirus, with a cumulative reduction of 1.04 Log.

For completeness, we will also describe a fractionation process that can give rise to a starting material, i.e. a biological sample (e.g. a partially purified immunoglobulin solution as used in the preferred embodiment described above).

In the above specific implementation example, the biological sample is a partially purified immunoglobulin solution derived from human plasma. The following description teaches how the inventors practiced the extraction of said immunoglobulin solution from frozen human plasma.

### EXAMPLE 2

### The Preparation of a Partially Purified Immunoglobulin Solution

Fraction II+III was prepared from frozen human plasma through cold ethanol precipitation (Cohn et al., J. Am. Chem. Soc. 1946; 68: 459-475). Briefly, the fraction containing immunoglobulin was obtained by cold ethanol precipitation of human plasma with 8% ethanol at pH 7.1, followed by another step of cold ethanol precipitation with 19% ethanol at pH 5.85. Centrifugation at 2,300×g separated the fraction II+III from the supernatant II+III. Then NaAc/HAc buffer (0.8 M/4M, pH 3.9) was added drop-wise to the re-dissolved fraction II+III solution to adjust pH to 5.1.95% ethanol was added drop-by-drop into the fraction II+III solution to a final ethanol concentration of 15%, which was further mixed gently for 1.5 h at −5 to −5.5° C., before centrifuged at 2,300×g to separate the resulting fraction from the resulting supernatant containing immunoglobulin.

### EXAMPLE 3

### The Production of Human Plasma Albumin that is Substantially Free from Infective Dengue Virus

Step 1: Fraction IV Precipitation

Supernatant II+III was obtained by two consecutive steps of cold ethanol precipitation, as described in Example 2. Afterwards, 95% ethanol was added drop-by-drop into said supernatant to a final ethanol concentration of 40%, which was further mixed gently for 1 hour at −5 to −5.5° C., before being centrifuged at 2,300×g to separate the fraction IV from the supernatant IV containing albumin.

Step 2: Pasteurization

The purified albumin solution was diafiltrated with 8 volumes of water and then concentrated to an albumin concentration of 22% with a 30 kD cut-off cassette (Millipore, Massachusetts, US). Pharmaceutical-grade sodium caprylate was added to the concentrated albumin solution to a final concentration of 0.032M, before adjustment of pH to 6.8 to make 20% albumin bulk. Subsequently, the 20% albumin in bulk (formulated with 0.032M sodium caprylate, pH 6.8) and the sterile-filtered albumin in final bottles were heated to 59° C. in a water bath for bulk pasteurization and terminal pasteurization, respectively. Gentle mixing with a mechanical stirrer (stainless steel) was applied to the bulk pasteurization. The time course of this treatment was 10 hours at 59-61° C.

The preferred embodiments of this second aspect of the present invention are thus fully described. Although the description referred to particular embodiments, it will be clear to one skilled in the art that the present invention may be practiced with variation of these specific details. Hence this invention should not be construed as limited to the embodiments set forth herein.

Quantification of Dengue Virus in Example 3.

In Example 3, the albumin solution prepared from frozen human plasma through cold ethanol precipitation was spiked with dengue virus at a ratio of 1:10. After the fractionation process, test samples were taken and titrated for quantity of dengue virus. The results are shown in Table 5.

In the subsequent step of pasteurization, the 20% albumin in bulk and the sterile-filtered albumin were spiked with dengue virus at a ratio of 1:20 and 1:25, respectively. Samples were taken out for virus titration throughout the time course of this treatment.

As listed in the Table 5, it was found that the albumin process is very effective in the inactivation of dengue virus, with a cumulative reduction of not less than 10.12 Log.

TABLE 5
Inactivation of Dengue Virus in the Albumin Process

| Process Step | Log Reduction of Dengue Virus |
|---|---|
| Fraction IV Precipitation | ≧5.18 |
| Bulk Pasteurization | ≧4.61 |
| Terminal Pasteurization | ≧4.94 |
| Cumulative | ≧10.12* |

Note:
*as ‘Bulk Pasteurization’ is procedurally similar to ‘Terminal Pasteurization’, only the latter is included in the ‘Cumulative’.

The pasteurization steps quickly inactivated dengue virus and no dengue virus was detected after five minutes of the heat treatment. Table 6 shows the Log reduction of dengue virus during the time course of these pasteurization steps.

TABLE 6
Inactivation of Dengue Virus during Pasteurization

Log Reduction of
Dengue Virus

| Pasteurization | Terminal | Bulk |
|---|---|---|
| 5-minute | ≧3.64 | ≧3.31 |
| 20-minute | ≧3.64 | ≧3.31 |
| 1-hour | ≧4.94 | ≧4.61 |
| 10-hour | ≧4.94 | ≧4.61 |

Note:
no virus was detected in the heat-treated samples, difference in Log reduction numbers at different time points is a reflection of sample volume assayed.

What is claimed is:

1. A method for inactivating and removing dengue virus from a biological sample containing at least one biomolecule of interest, said method comprising the steps of:
(a) treating the biological sample with at least one solvent under conditions sufficient to inactivate dengue viruses; and
(b) separating said virus from said biomolecule by cation exchange chromatography

2. The method of claim 1, wherein said cation exchange chromatography uses a medium selected from the group consisting of: silica, alumina, titania, cross-linked dextran, agarose, cross-linked agarose and a polymer derivatized with a cationic group.

3. The method of claim 1, wherein said cation exchange chromatography uses a medium of cross-linked agarose attached to carboxy methyl groups; and said step (b) comprises loading and equilibrating said column at pH 4.0±0.5; washing said column at pH 7.0±0.5; and eluting the biomolecule of interest using 0.1M glycine plus 0.15M NaCl at pH 9.0±0.5.

4. The method of claim 1, wherein said solvent contains at least one of the agents selected from the group consisting of: dialkylphosphates, trialkylphosphates, tri-(n-butyl)phosphate, tri-(t-butyl)phosphate, tri-(n-hexyl)phosphate, tri-(2-ethylhexyl)phosphate, and tri-(n-decyl)phosphate.

5. The method of claim 1, wherein said solvent is used in combination with a nonionic detergent, said detergent selected from the group consisting of: octylphenoxypolyethoxyethanol nonionic surfactant, octyl β-thioglucopyranoside and sorbitan mono-9-octadecenoate poly(oxy-1,1-ethanediyl).

6. The method of claim 5, wherein said solvent is tri-(n-butyl)phosphate and said detergent is octylphenoxypolyethoxyethanol nonionic surfactant.

7. The method of claim 6, wherein said detergent is used at a concentration of 1±0.1% and said solvent is used at a concentration of 0.3±0.1%.

8. The method of claim 1, wherein said conditions in step (a) are 28-32° C. for at least 1 hour.

9. The method of claim 1, further comprising a step of filtering with a 0.22 μm or 0.1 μm filter, followed by ultrafiltrating with a 35 nm filter.

10. The method of claim 1, wherein said biological sample is selected from the group consisting of: plasma, serum, a precipitate of plasma, a precipitate of serum, a cryoprecipitate of plasma, and a cryoprecipitate of serum.

11. The method of claim 1, wherein said biomolecule of interest is immunoglobulin.

12. The method of claim 1, wherein said biological sample is human immunoglobulin, said immunoglobulin being obtained from frozen human plasma, using the sequential steps:
(a) precipitating said plasma with 8±0.5% ethanol at pH 7.1±0.1;
(b) precipitating the supernatant obtained from step (a) with 19±0.5% ethanol at pH 5.85±0.05;
(c) re-dissolving the precipitate obtained from step (b);
(d) adjusting the pH to 5.1±0.1 with NaAc/HAc buffer of 0.8±0.05M/4±0.1M and pH 3.9±0.1;
(e) adding ethanol to a final ethanol concentration of 15±1.0%;
(f) mixing gently for 1 to 2 hours, at a temperature of −5 to −5.5° C.; and
(g) removing precipitates by centrifuging at 2,300×g to obtain a supernatant.

13. A method for inactivating and removing dengue virus from an immunoglobulin solution, said method comprising the steps of:
(a) treating the immunoglobulin solution with a mixture of octylphenoxypolyethoxyethanol nonionic surfactant and tri-(n-butyl)phosphate;
(b) removing said virus from said biomolecule through cation exchange chromatography using a medium comprising cross-linked agarose attached to carboxylmethyl groups, said removing steps comprise:
(i) equilibrating said column to pH 4.0±0.5,
(ii) washing said column with glycine at pH 7.0±0.5, and
(iii) eluting the biomolecule of interest using 0.1M glycine and 0.15M NaCl at pH 9.0±0.5.

14. The method of claim 13, wherein said octylphenoxypolyethoxyethanol nonionic surfactant is used at a concentration of 1±0.1% and said tri-(n-butyl)phosphate is used at a concentration of 0.3±0.1%; and said immunoglobulin solution is treated for a duration of at least 1 hour, at 28-32° C.

15. The method of claim 14, wherein said duration is at least 4 hours.

16. The method of claim 15, wherein said duration is 4 to 16 hours.

17. A method for producing human plasma albumin that is substantially free from infective dengue virus, comprising sequential steps:
(a) precipitating human plasma with 8±0.5% cold ethanol at pH 7.1±0.1;
(b) precipitating the supernatant obtained in step (a) with 19±0.5% ethanol at pH 5.85±0.05;
(c) precipitating the supernatant obtained in step (b) with 40±0.5% ethanol at pH 5.86±0.05;
(d) precipitating the supernatant obtained in step (c) with 40±0.5% ethanol at pH 4.77±0.05,
(e) diafiltrating the precipitate to make a solution of 20% albumin;
(f) adding 0.032M sodium caprylate of pH 6.8 to said albumin; and
(g) heating the resulting mixture to 59-61° C. for a duration of at least 1 hour.

18. The method of claim 17, wherein said duration in step (g) is 10 hours.

19. The method of claim 17, wherein said diafiltration in step (e) comprises ultrafiltration.
